(19) United States
(12) Patent Application Publication
(10) Pub. No.: US 2004/0015191 A1
(43) Pub. Date: Jan. 22, 2004

(54) Capturing images of a defibrillation scene

(76) Inventors: Alejandro A. Otman, Bothell, WA (US); Gregory T. Kavounas, Kirkland, WA (US); Richard C. Nova, Kirkland, WA (US); Mark P. Moore, Redmond, WA (US)

Correspondence Address: SHUMAKER & SIEFFERT, P. A., 8425 SEASONS PARKWAY, SUITE 105, ST. PAUL, MN, 55125, US

(21) Appl. No.: 10/405,071

(22) Filed: Mar. 31, 2003

Related U.S. Application Data
(60) Provisional application No. 60/385,019, filed on May 31, 2002.

(51) Int. Cl. A61N 1/39

(52) U.S. Cl. 607/5

(57) ABSTRACT

Techniques for capturing images of a defibrillation scene may involve obtaining images of a defibrillator scene using a camera coupled, in communication with, or integrated with an external defibrillator, such as an AED. For example, when a person arrives on the scene of a patient suffering sudden cardiac arrest (SCA) with a defibrillator, the person aims the camera at the patient and obtains an image of the patient. The camera may obtain a series of images, such as video imagery, and store the images in a storage medium associated with the camera or the defibrillator. The stored images may serve to document the defibrillation incident for later evaluation. For example, the stored images may provide a video record of actions taken, observed patient responses, and other significant events during the course of the incident. In some instances, the camera or defibrillator may transmit the images to a remote assistance center.

This application claims priority from U.S. provisional application serial No. 60/385,019, filed May 31, 2002, the entire content of which is incorporated herein by reference.

### FIELD

The invention relates to medical devices, and more particularly, to medical devices that monitor or treat cardiac emergencies.

### BACKGROUND

A sudden cardiac arrest (SCA) incident can kill a victim. More aptly called sudden cardiac death, sudden cardiac arrest is a condition in which the heartbeat stops suddenly and unexpectedly. It is caused by life-threatening arrhythmias, which are abnormalities in the heart's electrical system. The most common arrhythmia is ventricular fibrillation. In this condition, the heart beats too chaotically to be able to pump blood to the body and brain.

SCA is one of the leading causes of death among American adults, killing approximately 225,000 people a year. Two out of every three deaths happen outside of the hospital. SCA is unpredictable, and can happen to anyone, anywhere—even to a child. Risk increases with age. Although pre-existing heart disease is a common cause of cardiac arrest, many victims have never had any heart problems.

An SCA event is to be distinguished from a heart attack, although a person suffering a heart attack is more likely to develop abnormal heart rhythms and SCA. In contrast to SCA, a heart attack is caused by blocked blood flow to the heart muscle so that the muscle begins to die. SCA, on the other hand, is caused by an abnormal heart rhythm. Also, a heart attack is often preceded by chest, arm, upper abdomen, or jaw pain. Nausea and sweating are common. There is rarely a warning before sudden cardiac arrest.

Furthermore, while heart attack patients usually remain conscious, SCA victims always lose consciousness. More particularly, a victim of SCA first loses his or her pulse, then consciousness, and finally the ability to breathe. Without immediate treatment, the victim almost always dies.

Therefore, treatment must be administered as soon as possible to increase chances of survival. In the initial few minutes of the SCA incident, the probability of survival diminishes by 10% for every minute. Responding to an incident usually requires specially trained paramedics and equipment. The equipment may include an Automated External Defibrillator (AED). The non-medical public at large is slowly starting to appreciate that AEDs can be purchased and installed in places where many people congregate, or places otherwise hard to reach for medical personnel, such as airplanes.

### SUMMARY

The invention is directed to techniques for capturing images of a defibrillation scene. The techniques may involve obtaining images of a defibrillator scene using a camera. The camera may be coupled, in communication with, or integrated with a defibrillator, such as an AED. For example, when a person arrives on the scene of a patient suffering SCA with a defibrillator, the person aims the camera at the patient and obtains an image of the patient.

The camera may capture a series of images, such as video imagery, and store the images in a storage medium associated with the camera, the defibrillator, or both. The stored images may serve to document the defibrillation incident for later evaluation. For example, the stored images may provide a video record of actions taken, observed patient responses, and other significant events during the course of the incident.

In some instances, the camera or defibrillator may transmit the images to a remote assistance center via a communication link. The communication link may support two-way communication with the remote assistance center. In this manner, a trained person at the remote assistance center staff can provide rescue instructions and feedback to the person operating an AED.

The images may be stored or transmitted with other operational data concerning the performance of the defibrillator or the course of the defibrillation incident. For example, the operational data may include data concerning actions taken, observed patient responses, and other significant events during the course of the incident. The operational data also may include information concerning delivered pulses, such as energy level, pulse width, amplitude, or shape. The operational data also may include a digital representation of the ECG for a patient. Further, the operational data may include patient information such as patient name, address, and vital statistics, as well as the date, time and location of the defibrillation incident. The operational data may be recorded in a coordinated manner with the images.

In one embodiment, the invention provides a method comprising obtaining an image of a patient, and storing the image in a defibrillator.

In another embodiment, the invention provides a method comprising obtaining an image of a patient, establishing a communication link with a remote defibrillation assistance center, and transmitting the image to the remote defibrillation assistance center via the communication link.

In an added embodiment, the invention provides a defibrillator comprising a defibrillator circuit, and a camera to obtain an image of a patient.

In another embodiment, the invention provides a device comprising means for obtaining an image of a patient, and means for storing the image in an external defibrillator.

In a further embodiment, the invention provides a method comprising obtaining an image of a patient from an external defibrillator, obtaining operational data associated with treatment of the patient from the external defibrillator, and presenting the image and the physiological data together on a display device.

In another embodiment, the invention provides a method comprising receiving an image of a defibrillation scene, and transmitting signals to control a defibrillator at the defibrillation scene.

The invention may provide a number of advantages. For example, the invention supports an alternative mode for documenting events during the course of a defibrillation emergency. In particular, a defibrillator may store or transmit still images or video imagery, and optionally audio or other data, to enable later evaluation of measures taken during the course of the rescue attempt, and assessment of the state and responsiveness of the patient to particular activities. In addition, the transmission of images may facilitate two-way communication with a remote assistance center, and permit trained personnel to offer instructions for defibrillator operation or actively control defibrillator operation.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram illustrating administration of an AED to a defibrillation patient.

FIG. 2 is a diagram illustrating administration of a camera-equipped AED in accordance with an embodiment of the invention.

FIG. 3 is a diagram illustrating the camera-equipped AED of FIG. 2 in further detail.

FIG. 4 is a block diagram illustrating exemplary system components of the camera-equipped AED of FIG. 3 in further detail.

FIG. 5 is a flow diagram illustrating a method for capturing images of a defibrillation scene.

FIG. 6 is a flow diagram illustrating the uploading of information captured by a camera-equipped AED to a medical informatics system.

FIG. 7 is a diagram illustrating an example user interface screen for a medical informatics system that stores images of a defibrillation scene.

FIG. 8 is a diagram illustrating a communication link between a camera-equipped AED and a remote assistance center.

FIG. 9 is a flow diagram illustrating a method for communication between a camera-equipped AED and a remote assistance center.

FIG. 10 is a diagram illustrating attachment of a camera to an AED.

FIG. 11 is a diagram illustrating operation of a detached camera with an AED.

FIG. 12 is a diagram illustrating an AED with an integrated camera.

FIG. 13 is a diagram illustrating an AED coupled to a detached camera.

### DETAILED DESCRIPTION

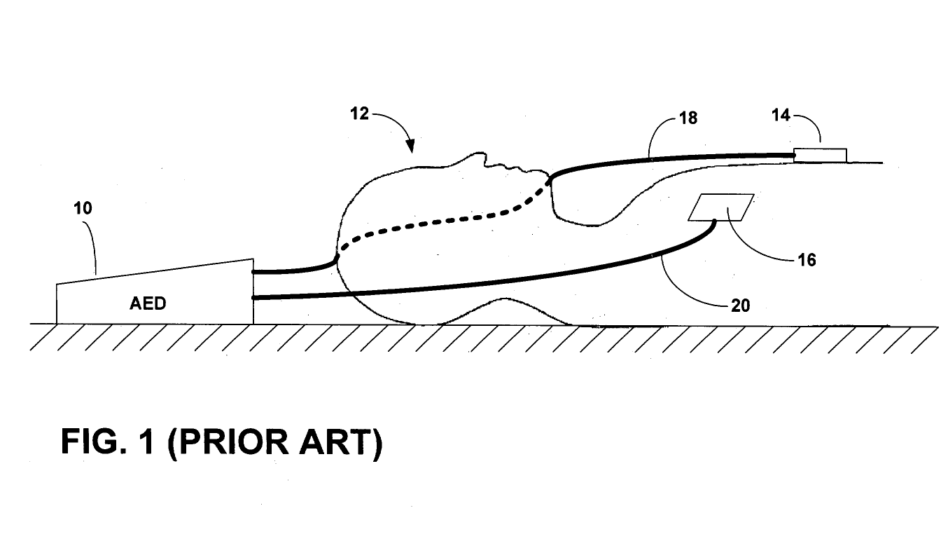

FIG. 1 is a diagram illustrating administration of an AED 10 to a defibrillation patient 12. AED 10 delivers defibrillation shocks to patient 12 via electrodes 14, 16. Electrodes 14, 16 are coupled to AED 10 via electrical leads 18, 20, respectively. AED 10 may be used by an untrained person to rescue a patient 12 suffering from SCA. Accordingly, AED 10 may be stored in a public place, such as an office, shopping area, sporting venue, airport, or the like. In general, AED 10 offers simplified operation and may present audible or visible prompts to instruct the user concerning operation.

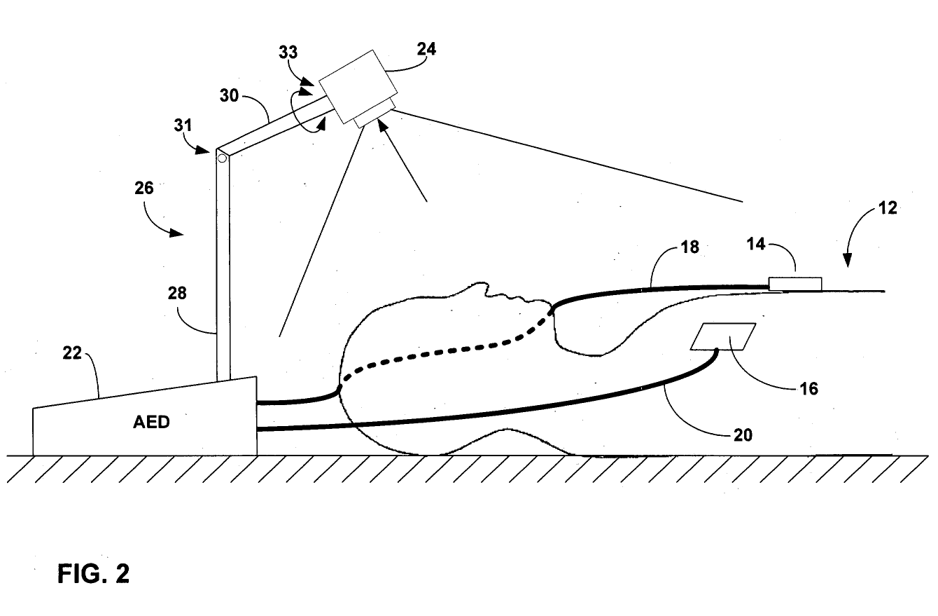

FIG. 2 is a diagram illustrating administration of a camera-equipped AED 22 in accordance with an embodiment of the invention. AED 22 may otherwise conform substantially to AED 10 of FIG. 1. AED 22 further includes, however, a camera 24 to capture images of the defibrillation scene. Although an AED 22 is described for purposes of example, the invention may be useful with other non-automated or minimally-automated external defibrillators, such as those used by paramedics and other medical personnel. The images obtained by camera 24 may include still images, video imagery or both. In addition, the images may be accompanied by contemporaneous audio obtained at the defibrillation scene. Camera 24 may be integrated with AED 22, coupled to the AED or completely detached from the AED.

In the example of FIG. 2, camera 24 may be mounted on a boom 26 with boom arms 28, 30. Camera 24 may be positioned, for example, by adjusting boom arms 28, 30 about a pivot point 31 or rotating camera 24 another pivot point 33 about boom arm 30. However, camera 24 may be positioned via a variety of positioning mechanisms, including telescoping or “gooseneck” mounting arms that allow flexible positioning. In addition, camera 24 may be positioned by automated positioning mechanisms that include motors to actuate the boom arms 28, 30 or other positioning components to automatically move and position the camera. Accordingly, the positioning mechanisms illustrated in FIG. 2 are merely for purposes of illustration and should not be considered limiting of the invention as broadly embodied and described herein. For example, camera 24 may be embedded within AED 22, coupled to the AED via a flexible electrical or optical connector, coupled by a wireless link, or the like.

In general, a person using AED 22 may obtain an image of the defibrillation scene and, particularly patient 12, using camera 24. When a person arrives on the scene of a patient 12 suffering SCA with AED 22, the person aims the camera at the patient and obtains an image of the patient. AED 22 may store the image obtained by camera 24. Alternatively, AED 22 may transmit the obtained image directly to a remote assistance center. The remote assistance center may be, for example, an emergency services center staffed by trained medical personnel. The image may be stored or transmitted along with operational data obtained by AED 22. In this manner, physicians or other personnel may make their own remote assessments of patient condition. In addition, in the event two-way communication is provided, a physician can query a patient or AED operator through the existing communication link and hear and see patient response via a remote computer workstation. In this manner, the physician can evaluate the extent of trauma and impact that can be seen via the images sent from the defibrillation scene. Accordingly, some embodiments of the invention may be especially suitable for placement in airplanes to permit remote evaluation and minimize unnecessary flight diversions.

Camera 24 may obtain a series of images, such as still photos or video imagery. The stored images may serve to document the defibrillation incident for later evaluation. For example, the stored images may provide a video record of actions taken, observed patient responses, and other significant events during the course of the incident. The operational data may store similar information in a non-image format, e.g., as text, codes, marker channel events, or the like.

In some instances, camera 24 or AED 22 may transmit the images to a remote assistance center (not shown in FIG. 2) in real-time via a communication link. The communication link may support two-way communication with the remote assistance center, permitting trained personnel at the remote assistance center staff to provide rescue instructions and feedback to the person operating AED 22. In some cases, the remote assistance center may issue control signals for direct control of defibrillator 22. In particular, the communication link may permit the remote assistance center to control defibrillation shocks and shock parameters such as shock levels. In addition, the images may be stored or transmitted to the remote assistance center for subsequent replay. The images may by used for a variety of purposes, e.g., to administer additional therapy to the patient, for training purposes, and the like.

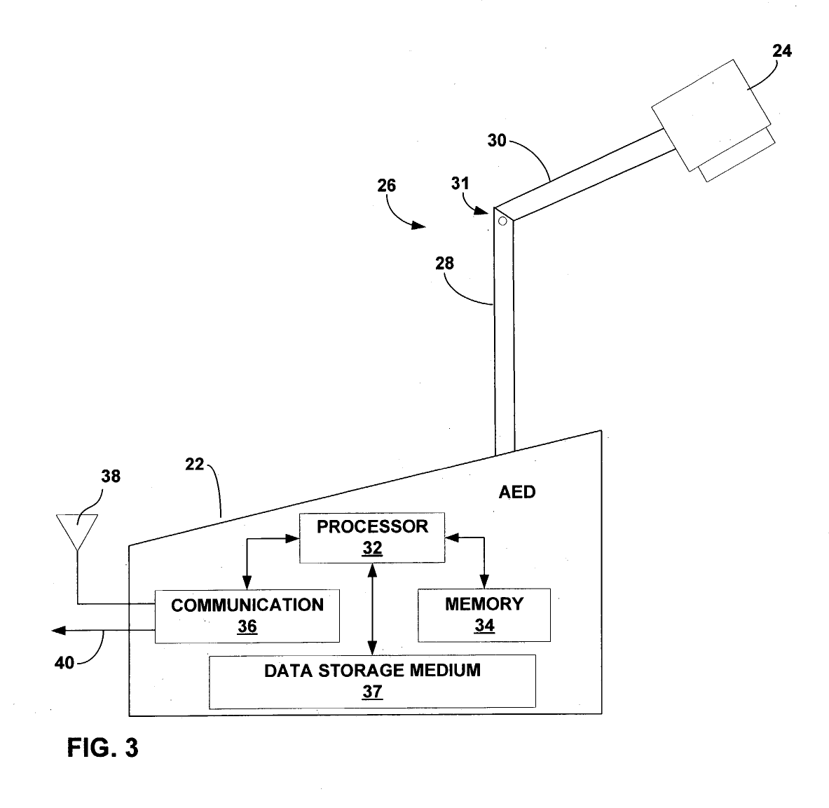

FIG. 3 is a diagram illustrating an exemplary embodiment of the camera-equipped AED 22 of FIG. 2 in further detail. As shown in FIG. 3, AED 22 may include a processor 32, a memory 34, a communication module 36, and a data storage medium 37. Processor 32 executes instructions stored in memory 34 to drive the operation of AED 22. For example, processor 32 may control a stimulation interface (not shown in FIG. 3) to deliver shocks to patient 12. In addition, processor 32 may drive a user interface to receive user instructions and present feedback to the user.

Data storage medium 37 may store images obtained by camera 24, as well as other information such as operational information, if desired. Data storage medium 37 may take a variety of forms, including solid state, magnetic or optical media. As an example, AED 22 may include a magnetic hard drive to store the substantial amount of data typically associated with digitized imagery, or a removable storage medium such as an optical or magnetic disk drive that allows the images to be manually transferred. Moreover, although illustrated for exemplary purposes as separate storage media, memory 34 and data storage medium 37 may comprise a single storage medium, or two or more storage media.

Alternatively, camera 24 may store images locally within a data storage medium provided in the camera. In this case, camera 24 may store captured imagery on magnetic tape, magnetic disk, optical disk, solid state media or the like. AED 22 may control camera 24, e.g., via a USB, IEEE 1394, or other connection, to capture and store images and also control the camera to stream or otherwise transfer the images to AED 22 for transmission to a remote assistance center or some other archival site. In still other embodiments, a communication module similar to communication module 36 may be provided within camera 24 itself to support transmission of images directly from the camera to a remote assistance center.

As a further alternative, AED 22 may transmit the images directly to a remote device without substantial intermediate storage of image data within the AED. For example, communication module 36 may transmit information, including images, operational data, or both, via a wireless radio frequency (RF) antenna 38 or a wired network connection 40. In particular, communication module 36 may transmit the information to a remote assistance center. In some embodiments, communication module 36 may provide a mobile wireless telephone interface to contact the remote assistance center. In this case, communication module 36 may automatically dial a telephone number stored within AED 22 to contact the remote assistance center.

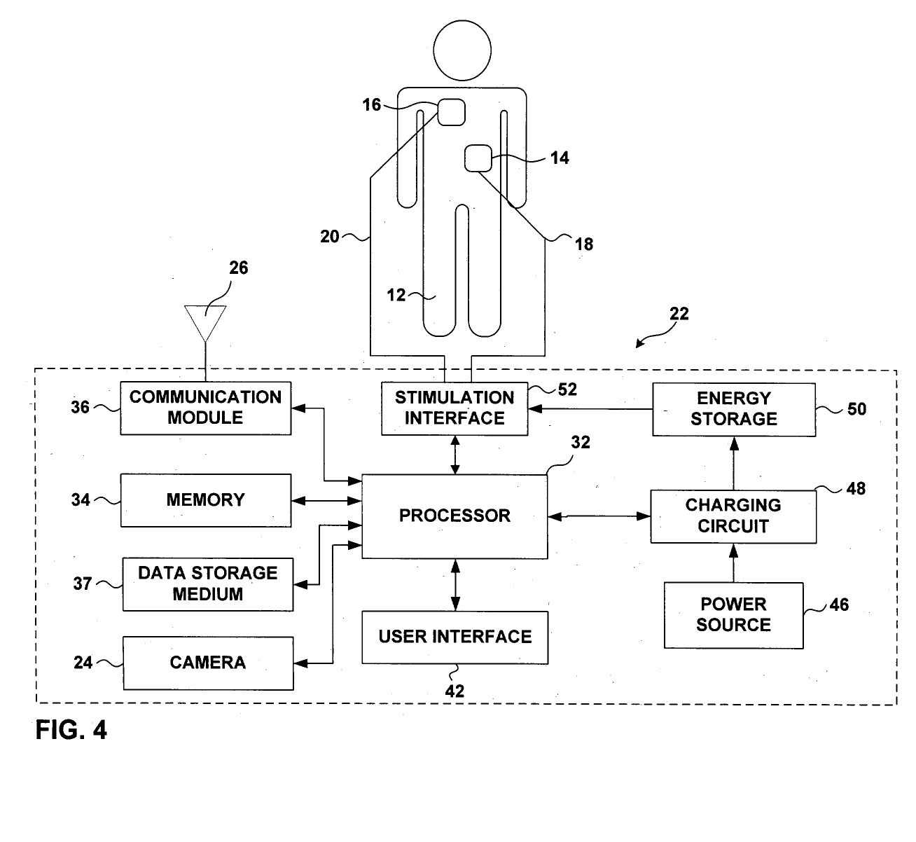

FIG. 4 is a block diagram illustrating exemplary components of the camera-equipped AED 22 of FIG. 3 in further detail. As shown in FIG. 4, AED 22 may include a variety of components such as electrodes 14, 16, leads 18, 20, camera 24, processor 32, memory 34, communication module 36, storage device 37, user interface 42, power source 46, charging circuit 48, energy storage unit 50 and stimulation interface 52.

In general, processor 32 controls the operations of AED 22 to deliver defibrillation pulses to patient 12 and monitor patient activity. Processor 32 also may interact with camera 24 and data storage medium 37 to process and- store images captured by the camera. Alternatively, a separate video processor may be provided in AED 22 for this purpose. Processor 32 also may interact with communication module 36 to transmit images, and optionally other information, to a remote assistance center. In addition, if two-way communication is provided, processor 32 may receive information including, in some embodiments, control signals from the remote assistance center.

Processor 32 may, for example, take the form of a microprocessor, microcontroller, or an application specific integrated circuit (ASIC), field programmable logic array (FPGA), or other equivalent discrete or integrated logic circuitry. Memory 34 is accessible by processor 32, and may include program instructions that cause the processor to perform the functions attributed to the processor herein. Memory 34 may take the form of random access memory. (RAM) or read-only memory (ROM) containing program instructions that cause processor 32 to monitor patient 12, deliver defibrillation pulses to the patient, and generate medical event information during the treatment of the patient.

AED 22 is coupled to patient 12 in order to facilitate the treatment of the patient, e.g., sensing electrical activity within the heart of patient 16 and delivering defibrillation pulses to patient 16. AED 22 is coupled to patient 12 via electrodes 14, 16 and leads 18, 20. Electrodes 14, 16 may include hand-held electrode paddles or adhesive electrode pads placed on the skin of patient 12. AED 22 senses electrical activity of the heart of patient 12 and delivers defibrillation pulses to the patient electrodes 14, 16 and leads 18, 20. Electrodes 14, 16 and leads 18, 20 are coupled to defibrillator 12 via a stimulation interface 52. In a typical application, stimulation interface 52 includes sockets to receive leads 18, 20.

Stimulation interface 52 may include a switch (not shown in FIG. 4) that, when activated, couples an energy storage unit 50 to leads 18, 20. Energy storage unit 50 stores the energy to be delivered to patient 12 in the form of a defibrillation pulse. The switch may be of conventional design and may be formed, for example, by electrically operated relays that coupled energy storage unit 50 to one or both of leads 18, 20. Alternatively, the switch may comprise an arrangement of solid-state devices such as silicon-controlled rectifiers or insulated gate bipolar transistors.

Energy storage unit 36 includes components, such as one or more capacitors, that store the energy to be delivered to patient 12 via electrodes 14, 16 and leads 18, 20. Before a defibrillation pulse may be delivered to patient 12, energy storage unit 50 must be charged. Processor 32 directs a charging circuit 48 to charge energy storage unit 50 to a high voltage level. Charging circuit 48 comprises, for example, a flyback charger that transfers energy from a power source 46 to energy storage unit 50.

As mentioned above, in some embodiments, the components of FIG. 4 may alternatively be arranged to form a manual, i.e., non- or minimally automated, defibrillator rather than an AED. Where the defibrillator is a manual defibrillator, an operator using the defibrillator may select an energy level for each defibrillation pulse delivered to patient 12. Processor 32 may receive the selection made by the operator via a user interface 42, which may include input devices, such as a keypad and various buttons or dials, and output devices, such as various indicator lights, a CRT, LED, or LCD screen, and a speaker. Where the defibrillator 12 is an AED, processor 32 may select an energy level from a preprogrammed progression of energy levels stored in memory 34 based on number of defibrillation pulses already delivered to patient 12.

When the energy stored in energy storage unit 50 reaches the desired energy level, processor 32 controls user interface 42 to provide an indication to the AED operator that AED 22 is ready to deliver a defibrillation pulse to patient 12, such as an indicator light or a voice prompt. The defibrillation pulse may be delivered manually or automatically. Where the defibrillation pulse is delivered manually, the AED operator may direct processor 32 to deliver the defibrillation pulse via user interface 42 by, for example pressing a button. In either case, processor 32 activates the switch to electrically connect energy storage unit 50 to leads 18, 20, and thereby deliver the defibrillation pulse to patient 12 via electrodes 14, 16.

Processor 32 may modulate the defibrillation pulse delivered to patient 12. Processor 32 may, for example, control the switch to regulate the shape of the waveform of the pulse and the width of the pulse. Processor 32 may control the switch to modulate the pulse to, for example, provide a multiphasic pulse, such as a biphasic truncated exponential pulse, as is known in the art.

Processor 32 may perform other functions as well, such as monitoring electrical activity of the heart of patient 12 sensed via electrodes 14, 16 and received via stimulation interface 52. Processor 32 may determine whether the heart of patient 12 is fibrillating based upon the sensed electrical activity in order to determine whether a defibrillation pulse should be delivered to the patient. Where a defibrillation pulse has already been delivered, processor 32 may evaluate the efficacy of the delivered defibrillation pulse by determining if the heart is still fibrillating in order to determine whether an additional defibrillation pulse is warranted. Processor 32 may automatically deliver defibrillation pulses based on these determinations, or may advise the AED operator of these determinations via user interface 42. Processor 32 may display an electrocardiogram (ECG) based on the sensed electrical activity via user interface 42.

Processor 32 may store an indication of the time of delivery of each defibrillation pulse delivered to patient 12 as medical event information within data storage medium 37 for patient 12. Processor 32 may also store the energy level of each pulse and other characteristics of each pulse, such as the width, amplitude, or shape, as medical event information for patient 12. Processor 32 may also store a digital representation of the ECG as operational data for patient 12. Further, the operational data may include patient information such as patient name, address, and vital statistics, as well as the date, time and location of the defibrillation incident.

In this manner, processor 32 may obtain and store a record of operational data, as well as actions taken, observed patient responses, and other significant events during the course of the incident at the defibrillation scene. This information may take a variety of forms. With camera 24, AED 22 is capable of capturing images, such as still images or video imagery, at the defibrillation scene during the course of the incident. The captured imagery may be stored and then evaluated independently or in conjunction with other stored operational data.

The captured imagery, in some embodiments, may be correlated with particular patients and particular items of operational data. For example, images or video frames may be time-stamped and indexed to an existing or newly created patient record in AED 22. In addition, the images may be correlated using tables, pointers or the like to associate the images with particular items of operational data. For example, an image taken at the time a particular defibrillation shock is delivered may be correlated with operational data indicating the characteristics of the defibrillation shock, such as energy level, pulse width, amplitude and shape. In this case, the image and the operational data may be evaluated independently or together.

Also, in some embodiments, processor 32 may interact with communication module 36 to transmit the images, and optionally the operational data, to a remote assistance center. A trained person at the remote assistance center may view the images and offer instructions to the AED operator handling AED 22 via user interface 42, in the event communication module 36 provides two-way communication. The instructions may be audible or visual, or a combination of both. For example, in response to instructions from the remote assistance center, AED 22 may display text instructions. Alternatively, AED 22 may simply emit verbal instructions via a speaker provided by user interface 42. In addition, AED 22 may be configured to accept control signals attuned to control the AED, camera 24, or both, from the remote assistance center via communication module 36. Processor 32 may process the control signals to control operation of AED 22.

As a further option, user interface 42 may include a microphone (not shown) that detects sounds in the vicinity of defibrillator 12. The microphone can capture the voice of the AED operator. Processor 32 may receive signals from the microphone and transmit audio to the remote assistance center via communication module 36, facilitating two-way communication between the operator handling AED 22 and a person at the remote assistance center. Camera 24 may include its own microphone to capture audio in conjunction with still or video images of the defibrillation scene. In some cases, voice communication from the AED operator to the remote assistance center may be accomplished via the audio track accompanying capture video, particularly if communication module 36 transmits the video and audio in real-time.

Communication module 36 may be realized by a variety of communication devices. For example, communication module 36 may include a network card, a wireless local area network (WLAN) card, a mobile phone, an infrared (IR) card, a modem, a network-enabled PDA or mobile computer, or any combination thereof. Communication module 36 may couple AED 22 directly to a communication network, or indirectly to the network via a network access point. For example, communication module 16 may electrically couple AED 22 to a mobile phone via a connector. As an alternative, communication module 36 may electromagnetically couple AED 22 to a WLAN access point.

The communication network used by communication module 36 may be a combination of network architectures, including a public switched telephone network (PSTN), an integrated services digital network (ISDN), an Internet protocol (IP) network, a local area network (LAN), a wide area network (WAN), a wireless communications network, or an asynchronous transfer mode (ATM) network. In general, the communication network could be selected to provide sufficient bandwidth to support real-time transmission of either still images or video imagery.

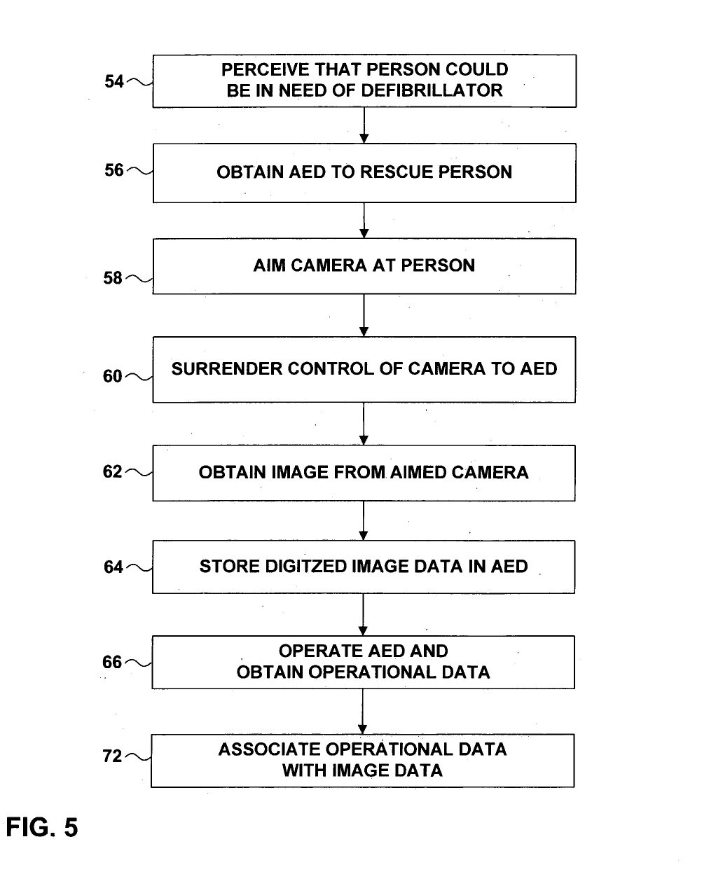

FIG. 5 is a flow diagram illustrating an example method for capturing images of a defibrillation scene. As shown in FIG. 5, upon perceiving that a patient is suffering SCA and could be in need of a defibrillator (54), a rescuer obtains an AED to rescue the patient (56). The rescuer, i.e., the AED operator, may be an untrained person who encounters the person suffering SCA, or a designated first-responder within a particular site, such as an office, shopping area, sporting venue, airport, or the like. According to the method of FIG. 5, the AED is equipped with a camera as described herein. As further indicated in FIG. 5, the AED operator aims the camera at the person suffering from SCA (58).

To aid in aiming the camera, the AED or the camera may provide a viewfinder, and optionally a display such as a liquid crystal display (LCD). Upon aiming the camera, the AED operator may surrender control of the camera to the AED (60). For example, the AED then takes control of the camera to acquire still images or video imagery of the patient, and optionally audio. Camera 24 may be any system or device capable of rendering the image of the defibrillation scene in digital form. Camera 24 may be either a still camera or a video camera, but is preferably a digital camera to provide immediate output of images in digital video form. Camera 24 may generate video output in any of a variety of formats, such as NTSC, PAL, SECAM, S-video or the like. Elements such as video rate, zooming, and the like may be controlled by AED 22, e.g., by processing prior images and determining where and how to look next. Alternatively, a sequence of settings may be preprogrammed within AED 22 for capturing images.

In some embodiments, AED 22 may surrender control of camera 24 to a remote assistance center. The remote assistance center may receive the images from the camera, and transmit control signals to control operations of the AED and operations of the camera, such as image capture, zoom and the like. In some embodiments, the camera and its positioning hardware may be automated to permit automated tilt, pan, zoom and the like.

Upon obtaining an image from the aimed camera (62), the AED may store a digitized image data file (64). In this manner, the AED may preserve an image-based record of the defibrillation scene. In embodiments, if a camera is attached to the AED, the AED may present a message to provide the AED operator with the option of using the camera. The AED operator may accept an image captured by the camera by entering user input. In response, the AED stores the image in the data storage medium carried by the AED or transmits the image directly to a remote assistance center. The user input may be entered by pressing areas on a touchscreen, pressing buttons, or actuating other similar input media.

In some cases, the AED may transmit the images directly to a remote assistance center, or some other destination for evaluation or archival. As the camera captures images, the user operates the AED to deliver a defibrillation shock to the patient and obtains operational data (66). Optionally, the AED may be operated remotely, e.g., in response to control signals transmitted by a remote assistance center. Again, operational data may refer to data concerning actions taken, observed patient responses, and other significant events during the course of the incident, as well as information concerning delivered pulses, such as energy level, pulse width, amplitude, or shape.

With further reference to FIG. 5, the AED may associate the operational data with captured image data (72), either indirectly via time stamps, or directly by file headers, tables, pointers, or the like. In this manner, the images may be evaluated independently or in conjunction with operational data. In some embodiments, as will be described, operational data may be presented with captured imagery to facilitate analysis of the course of events at a defibrillation scene.

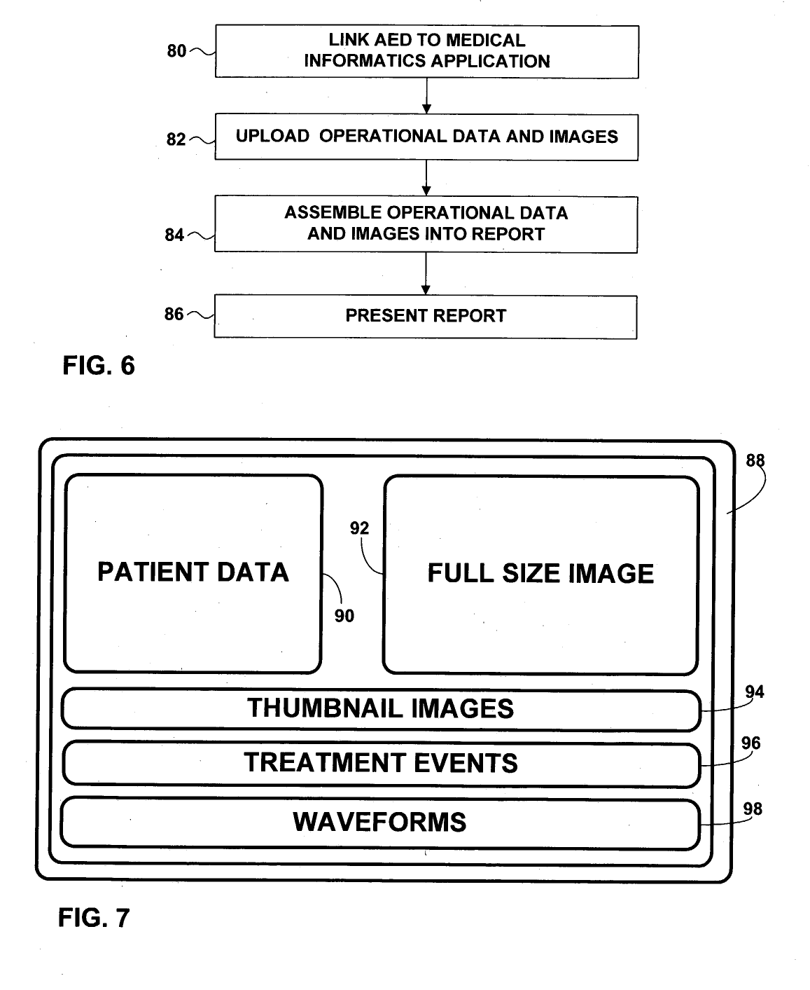

FIG. 6 is a flow diagram illustrating the uploading of information captured by a camera-equipped AED to a medical informatics system. As shown in FIG. 6, uploading information captured by the camera-equipped AED may involve linking the AED to a medical informatics application running on a computer workstation (80). The link between the AED and the medical informatics application can be made directly by coupling the AED to the computer workstation, e.g., a USB connection, IEEE 1394 connection or the like. Alternatively, the AED may interact with the computer workstation via a remote network connection. In either case, the medical informatics application may interrogate the AED to upload operational data and images (82).

The medical informatics application may assemble the operational data and images into a report (84) for a particular defibrillation incident involving a particular patient, and present the report (86) to a user for evaluation. Images and data may be merged by the medical informatics application to create an integrated patient record containing images, patient waveforms and treatment events. Again, the operational data and images may be correlated with one another using headers, tables, pointers and the like, so that the user may view the image and operational data in conjunction with one another. In some embodiments, specific operational data items may be correlated with specific images captured during the course of the defibrillation incident.

FIG. 7 is a diagram illustrating an example user interface screen 88 for a medical informatics system that stores images of a defibrillation scene. In general, user interface screen 88 may present a report that includes patient data 90, a full size image window 92, thumbnail images 94, treatment events 96 and waveforms 98. Patient data 90 may include patient name, address, and vital statistics, as well as the date, time and location of the defibrillation incident. Full size image window 92 may present a still image or video sequence with a size and resolution sufficient for adequate viewing of the defibrillation scene.

Thumbnail images 94 may present a set of low resolution versions of images captured by the AED during the course of the incident. By selecting a thumbnail image, the user directs the medical informatics application to present a larger, higher resolution version of the thumbnail image in full size image window 92. Each thumbnail image may pertain to a separate still image or a video sequence. For video sequences, the thumbnail may represent that captured video at the time of a significant event during the defibrillation incident, such as delivery of a defibrillation shock.

Treatment events 96 may set forth the times of particular events such as delivery of shocks and resulting physiological signals obtained by the AED to assess the patient response. In addition, treatment events 96 may set forth additional operational data concerning defibrillation shock energy levels, pulse widths, and the like. Waveforms 98 may include actual ECG information obtained by the AED, as well as marker channel information identifying particular events.

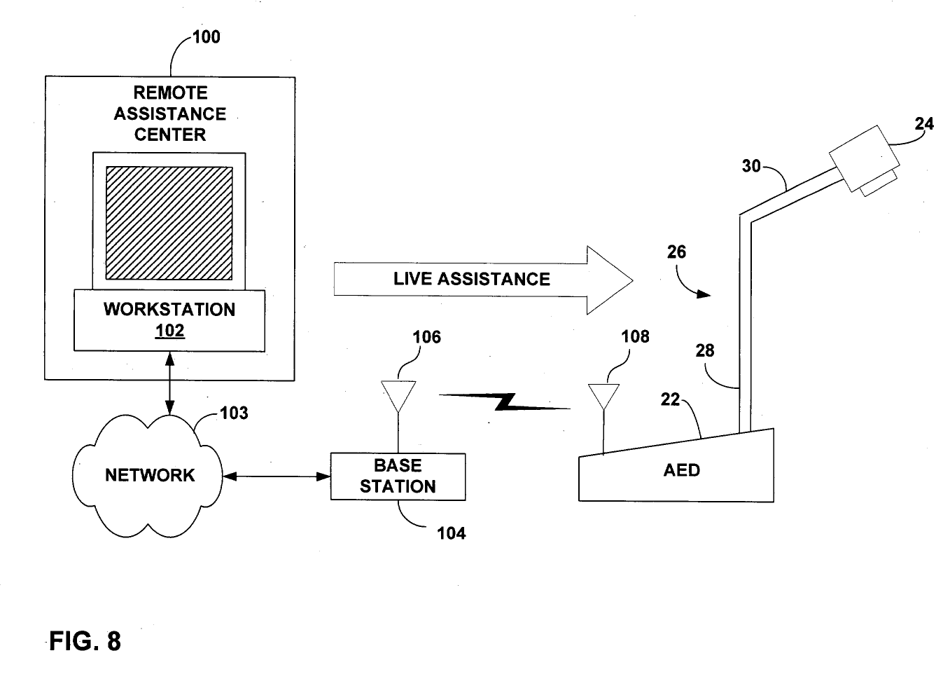

FIG. 8 is a diagram illustrating a communication link between a camera-equipped AED 22 and a remote assistance center 100. As shown in FIG. 8, remote assistance center 100 includes a computer workstation 102 that permits trained medical personnel to communicate via a network 103 with an AED operator handling AED 22. In particular, computer workstation 100 may receive images and operational data captured by AED 22. In the example of FIG. 8, AED 22 provides a wireless communication interface to a base station 104 via RF antennas 106, 108. In particular, AED 22 may integrate a wireless telephone interface, in which case base station 104 may be a cellular telephone base station.

A mobile telephone interface may facilitate communication in remote locations where network access is not otherwise available. In many locations, however, such as offices, shopping areas, sporting venues, airports, or the like, AED 12 may communicate via wireless local area networks. In either case, the communication linked between AED 22 and remote assistance center 100 permits trained medical personnel to view images captured by camera 24, as well as audio and other operational data. In addition, if two-way communication is provided, medical personnel at the remote assistance center 100 may transmit instructions to the operator handling AED 22 or control signals to control the operation of AED 22. In this manner, remote assistance center 100 may provide the AED operator with live assistance during the course of the defibrillation incident.

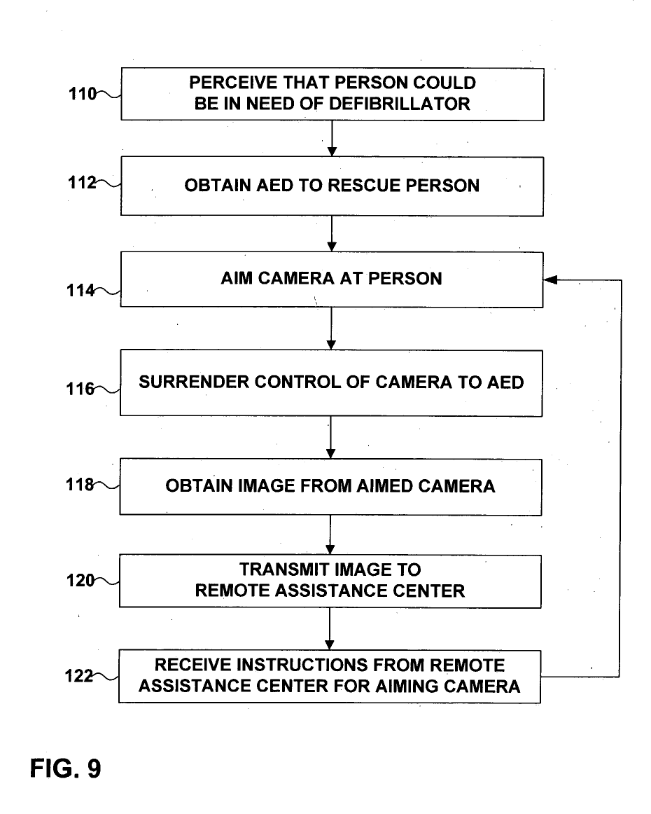

FIG. 9 is a flow diagram illustrating a method for communication between a camera equipped AED 22 and a remote assistance center 100. As shown in FIG. 9, upon perceiving that a patient could be in need of a defibrillator (110), a rescuer, i.e., an AED operator, obtains an AED to rescue the person (114). Upon aiming the camera at the person (114), the AED operator surrenders control of the camera to the AED (116). The AED obtains an image from the aimed camera (118), and transmits the image to the remote assistance center (120). Then, the AED operator receives instructions from the remote assistance center for aiming the camera (122). In this case, the remote assistance center may take over control of the AED by transmitting control signals for delivery of defibrillation shocks to the patient.

The remote assistance center can evaluate the situation for administering the therapy, and determine its effectiveness, and even request that the camera be aimed at a different place within the scene, as necessary. Furthermore, the person at the remote assistance center may iteratively instruct the AED operator for different use of the camera. In this way, the person at the remote assistance center can better evaluate what is happening at the defibrillation scene. For example, the person at the remote assistance center can evaluate the age and gender of the victim without having to ask any questions. This is especially useful if the person operating the AED is not trained, not well trained, or too overcome by the stress of the moment to communicate the situation.

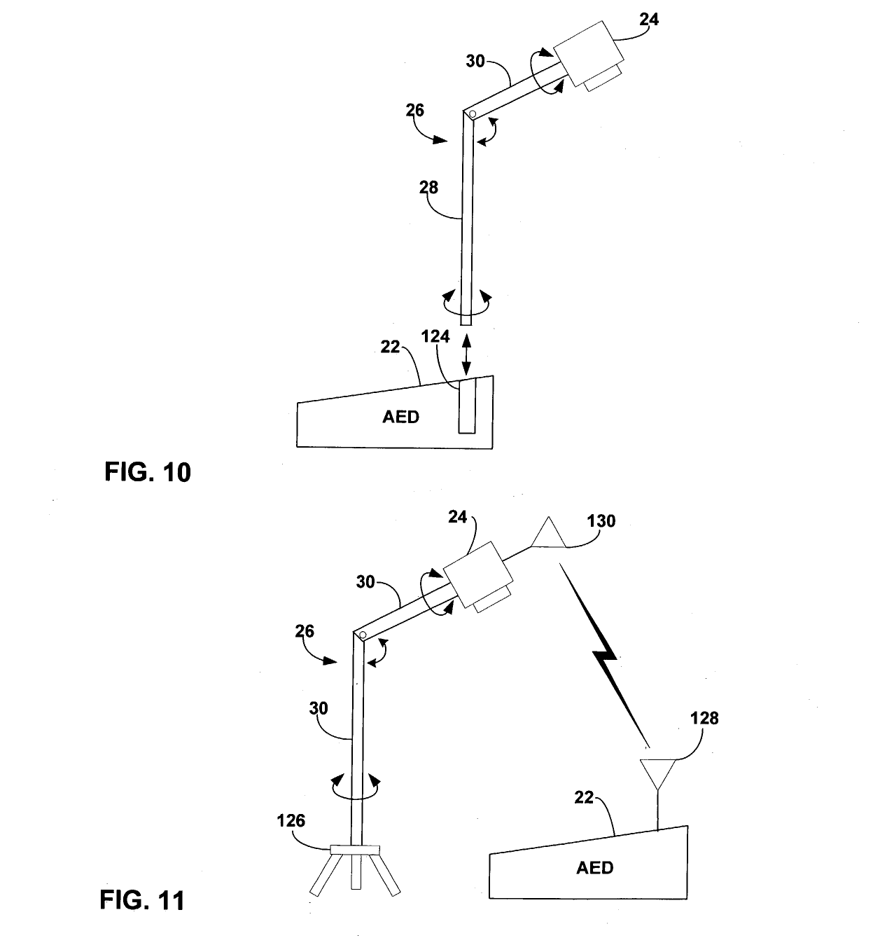

FIG. 10 is a diagram illustrating attachment of a camera 24 to an AED 22. In the example of FIG. 10, a positioning boom 26 associated with camera 24 may be mounted within a receptacle 124 in AED 22. In some embodiments, necessary electrical connections for communicating image data from camera 24 to AED 22 may extend through boom 26 and engage electrical terminals within receptacle 124. In addition, receptacle 124 may provide electrical terminals for delivery of electrical power to camera 24.

FIG. 11 is a diagram illustrating operation of a detached camera with an AED. In the example of FIG. 11, camera 24 is carried by a boom 26 mounted on a tripod 126. Accordingly, camera 24 may be positioned independently of AED 22. In this embodiment, camera 24 may carry its own power supply and provide a wireless transmitter for transmitting image data to AED 22 via RF antennas 128, 130. In this case, AED 22 may include a wireless receiver. Hence, AED 22 may include a wireless communication interface for communication with camera 24, in addition to a wireless or wired communication interface for communication with a remote assistance center. In other embodiments, camera 24 may be coupled to AED 22 via a wired connection. For example, a cable with one or more plugs may extend from camera 24 or tripod 126 for connection to a socket AED 22.

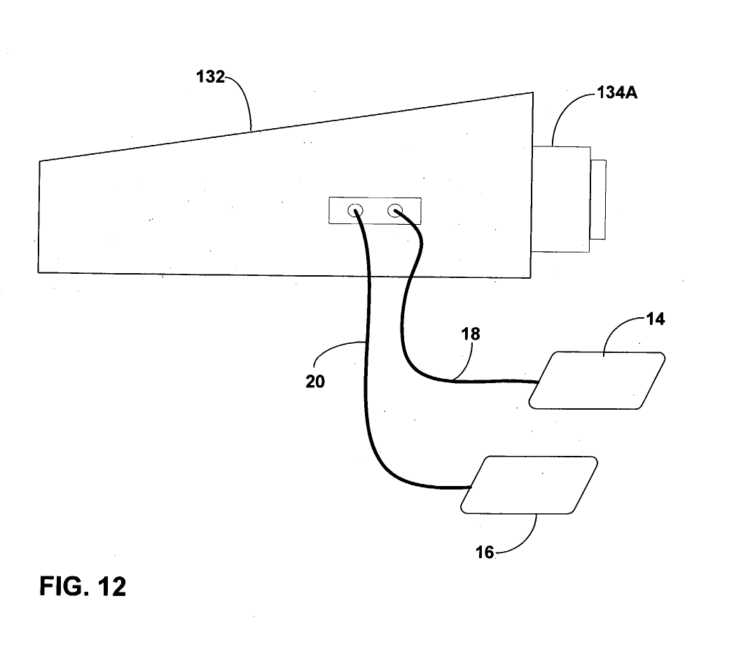

FIG. 12 is a diagram illustrating an AED 132 with an integrated camera 134A. In the example of FIG. 12, camera 134A may be mounted within a housing associated with AED 132, and may have a wide angle lens to capture images of the defibrillation scene. In this example, camera 134 and AED 132 may be connected internally, eliminating the need for a cable.

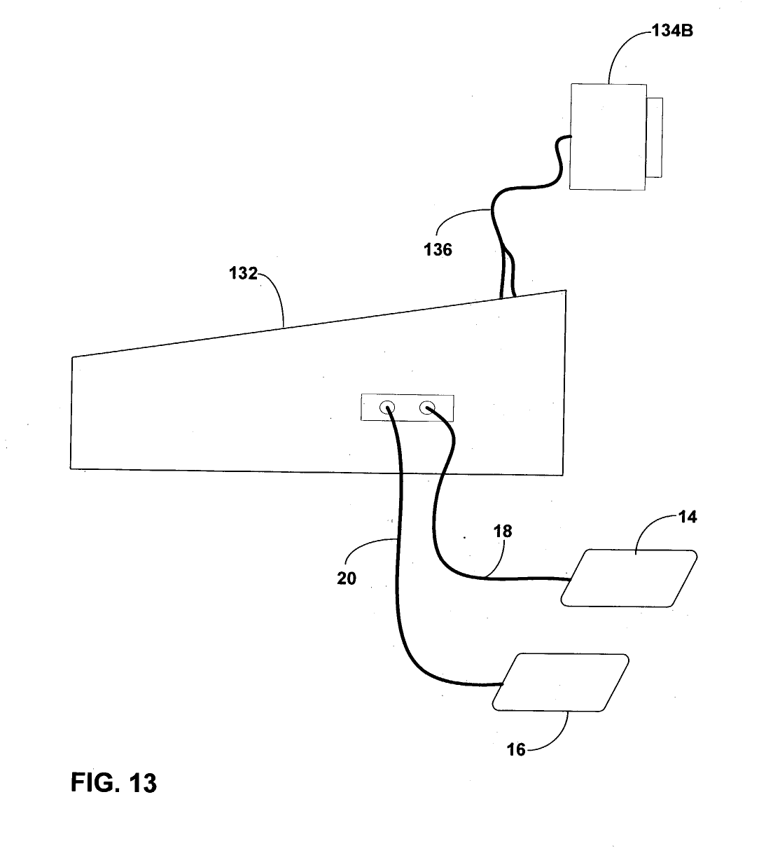

FIG. 13 is a diagram illustrating an AED 132 coupled to a detached camera 134B. In the example of FIG. 13, camera 134B is detached from AED 132 and includes electrical cable 136 that provides a wired connection to the AED. Hence, AED 134 may have a special “VIDEO IN” plug. The wired connection may support transport of image data to AED 132, and optionally power to camera 134B. In some embodiments, AED 132 may be configured to support camera 134B when the AED and camera are stored and therefore not in use.

A person skilled in the art will be able to practice the present invention in view of the description present in this document, which is to be taken as a whole. Numerous details have been set forth in order to provide a more thorough understanding of the invention. In other instances, well-known features have not been described in detail in order not to obscure unnecessarily the invention.

While the invention has been disclosed in its preferred form, the specific embodiments as disclosed and illustrated herein are not to be considered in a limiting sense. Indeed, it should be readily apparent to those skilled in the art in view of the present description that the invention may be modified in numerous ways. The inventor regards the subject matter of the invention to include all combinations and subcombinations of the various elements, features, functions and/or properties disclosed herein.

1. A method comprising:
obtaining an image of a patient; and
storing the image in a defibrillator.

2. The method of claim 1, further comprising associating the stored image with data about the patient.

3. The method of claim 1, further comprising:
obtaining operational data during operation of the external defibrillator; and
associating the operational data with the stored image.

4. The method of claim 1, further comprising communicating the stored image to another device.

5. The method of claim 3, in which the operational data includes one of physiological patient data and treatment event data.

6. The method of claim 1, further comprising obtaining a second image of the patient in the same way as the first.

7. The method of claim 1, further comprising:
obtaining the image via a camera; and
controlling operation of the camera via the external defibrillator.

8. The method of claim 1, further comprising obtaining the image using a camera, and surrendering control of the camera at least in part to the defibrillator.

9. The method of claim 1, further comprising obtaining the image using a camera, and transmitting the image from the camera to a remote assistance center.

10. The method of claim 9, further comprising transmitting the image from the camera to the remote assistance center via a communication module in the defibrillator.

11. The method of claim 1, further comprising obtaining the image using a camera, and surrendering control of the camera to a remote assistance center.

12. The method of claim 1, further comprising obtaining the image using a camera, and receiving instructions for aiming the camera from a remote assistance center.

13. The method of claim 1, further comprising obtaining the image using a camera, and receiving instructions for aiming the camera from the defibrillator.

14. A method comprising:
obtaining an image of a patient;
establishing a communication link with a remote defibrillation assistance center; and
transmitting the image to the remote defibrillation assistance center via the communication link.

15. The method of claim 14, further comprising:
reading a telephone number from a defibrillator; and
calling the telephone number to establish the communication link.

16. The method of claim 14, in which the communication link includes a link between the center and a defibrillator.

17. The method of claim 14, in which the communication link is a wireless telephone link established by the defibrillator.

18. The method of claim 14, further comprising receiving instructions from the center.

19. The method of claim 18, in which the instructions are verbal, and concern one of defibrillation and instructions about receiving further images.

20. The method of claim 18, in which the instructions are conveyed in signals attuned to operate one of a defibrillator and a camera that has been used to obtain the image.

21. A defibrillator comprising:
a defibrillator circuit to deliver a defibrillation shock to a patient; and
a camera to obtain an image of the patient.

22. The defibrillator of claim 21, further comprising a memory to store the image.

23. The defibrillator of claim 21, further comprising:
means for retransmitting the image to a remote defibrillation assistance center.

24. The external defibrillator of claim 21, further comprising a processor to associate the stored image with the patient.

25. The external defibrillator of claim 24, in which the processor obtains operational data during operation of the external defibrillator, and associates the operational data with the stored image.

26. The external defibrillator of claim 25, further comprising a communication circuit to communicate the operational data and the storage image to an external device.

27. The external defibrillator of claim 25, in which the operational data includes physiological patient data and treatment event data.

28. The external defibrillator of claim 24, further comprising a communication circuit to provide wireless communication between the camera and the processor.

29. The external defibrillator of claim 21, in which the camera is mounted to a housing associated with the external defibrillator.

30. The external defibrillator of claim 21, in which the camera is integrated with the external defibrillator.

31. The external defibrillator of claim 21, in which the camera is a video camera.

32. The external defibrillator of claim 21, further comprising obtaining a series of images of the patient during treatment of the patient with the external defibrillator.

33. The external defibrillator of claim 21, further comprising a processor to control operation of the camera.

34. The external defibrillator of claim 21, wherein the external defibrillator is an automated external defibrillator.

35. The external defibrillator of claim 21, further comprising a processor to control the defibrillator.

36. The external defibrillator of claim 21, further comprising a communication module to transmit the image to a remote assistance center.

37. The external defibrillator of claim 36, further comprising a processor that communicates with the remote assistance center via the communication module, the processor surrendering control of the camera to the remote assistance center.

38. The external defibrillator of claim 36, further comprising a processor to receive instructions for aiming the camera from the remote assistance center.

39. The external defibrillator of claim 36, further comprising a user interface to present instructions for aiming the camera.

40. A device comprising:
means for obtaining an image of a patient; and
means for storing the image in an external defibrillator.

41. The device of claim 40, further comprising associating the stored image with the patient.

42. The device of claim 40, further comprising:
means for obtaining operational data during operation of the external defibrillator; and
means for associating the operational data with the stored image.

43. The device of claim 42, further comprising means for communicating the operational data and the stored image to an external device.

44. The device of claim 42, in which the operational data includes physiological patient data and treatment event data.

45. A method comprising:
obtaining an image of a patient an external defibrillator;
obtaining operational data associated with treatment of the patient from the external defibrillator; and
presenting the image and the physiological data together on a display device.

46. The method of claim 45, in which the operational data includes physiological patient data and treatment event data.

47. The method of claim 45, in which the image includes video imagery.

48. The method of claim 45, in which the image includes a series of images of the patient during treatment of the patient with the external defibrillator.

49. A method comprising:
receiving an image of a defibrillation scene; and
transmitting signals to control a defibrillator at the defibrillation scene.

50. The method of claim 49, further comprising receiving the image at a remote assistance center.

51. The method of claim 49, in which the signals include signal to control delivery of a shock to a patient by the defibrillator.

52. The method of claim 49, in which transmitting signals includes:
establishing a communication link between the defibrillator and the remote assistance center; and
transmitting the image to the remote defibrillation assistance center via the communication link.

53. The method of claim 52, in which the communication link is a wireless telephone link established by the defibrillator.
